(19) United States
(12) Patent Application Publication
(10) Pub. No.: US 2011/0054481 A1
(43) Pub. Date: Mar. 3, 2011

(54) Delivery Containers and System

(76) Inventor: Michael Sproul, Breckenridge, CO (US)

(21) Appl. No.: 12/550,335

(22) Filed: Aug. 28, 2009

(51) Int. Cl. A61B 17/58

(52) U.S. Cl. 606/94

(57) ABSTRACT

A containment system for delivery of prepackaged substances to locations within a living individual that can be used in combination with existing cannulae used for this purpose is provided. The substance delivery containers are formed from a containment module; and a substance to be delivered into a specific location of a living individual disposed within the containment module. Prior to use, the containment module maintains a seal around the substance to prevent leakage and to isolate the substance from the surrounding environment. The containment module is formed from a material that is flexible and compressible and has structure and shape sufficient to define a distal end and a proximal end. The proximal end includes a shaped receiver for interacting with a tamp or similar pushing device for driving the containment module and/or the substance contained therein through a cannula to the desired location within the living individual.

### BACKGROUND OF THE INVENTION

This application relates to the delivery of substances into specific limited locations within a living individual, including human individuals.

Various medical procedures depend on the delivery of substances to specific and limited locations within living individuals. For example, vertebroplasty stabilizes collapsed vertebra using an acrylic bone cement which is injected through a needle and into the vertebral body. A related procedure known as kyphoplasty involves placement of a balloon into a collapsed vertebra, followed by injection of bone cement to stabilize the fracture.

In procedures of this type the substance to be delivered is frequently measured out or even made up by mixing component parts together in the operating room. This is time consuming, and introduces risk of error and wasted materials. Nevertheless, it remains commonplace, at least in part because of the variety of surgical tools available for performing procedures, and the fact that doctors have individual preferences for the tools to use.

US Patent Publication 2009/0069815 discloses a hollow tubular cartridge with a sharpened distal end and bone cement within the lumen of the cartridge for use in reinforcing bone, for example in the spine. The sharpened end of the cartridge is shown to be inserted directly into the spine. The bone cement may be pre-loaded in the cartridge.

US Patent Publication No. 2008/0119787 discloses a microsyringe for pre-packaged delivery of pharmaceuticals. It has a compressible chamber into which the pharmaceutical is pre-loaded, and a needle connected to this chamber via a flow control system. Thus, in this system the needle of the microsyringe used directly for delivery.

In order to avoid a requirement for doctors to change from their ordinary and familiar techniques and tools, it would be desirable to provide pre-packaged substances for delivery using these tools, for example via already existing and used catheters. Further, it would be desirable to have a pre-packaged option for such substances that did not require a change in tools between preparation steps, such as the formation of a cavity in cancellous bone, and the subsequent step of delivering substance to that cavity. The significant variety in these tools, both in terms of size and design, would seem to require a plethora of different sizes and shapes of containers for providing substances which is economically unfeasible and impractical.

### SUMMARY OF THE INVENTION

The present invention provides a containment system for delivery of prepackaged substances to locations within a living individual that can be used in combination with existing cannulae used for this purpose. In accordance with the invention, substance delivery containers comprise:
(a) a containment module; and
(b) a substance to be delivered into a specific location of a living individual disposed within the containment module.
   Prior to use, the containment module maintains a seal around the substance to prevent leakage and to isolate the substance from the surrounding environment.

The containment module is formed from a material that is flexible and compressible. Further, the containment module has structure and shape sufficient to define a distal end and a proximal end, said proximal end including a shaped receiver for interacting with a tamp or similar pushing device for driving the containment module and/or the substance contained therein through a cannula to the desired location within the living individual.

In some embodiments of the invention, the proximal end of the containment module comprises a lip which is larger in diameter than containment module, and which can interact with the proximal end of the cannula to prevent movement of the proximal end into the cannula. In some of these embodiments, the lip may be separable from the remainder of the containment module. In other embodiments, the lip is not separable and retains the containment module at the proximal end of the cannula. In the latter case, the distal end of the containment module optionally contains predefined locations at which the containment module will break in response to pressure to create an opening through which the substance may be expressed.

In some embodiments of the invention, the containment module is formed from a resorbable material.

In some embodiments of the invention the containment module has extensions on the exterior surface thereof between the proximal end and the distal end to limit lateral movement of the containment module when inserted into cannulae having different internal diameters.

In some embodiments of the invention the containment module has external surface structure that limits rotation of the containment module within a cannula.

In some embodiments of the invention, the containment module has volumetric measurements inscribed thereon to allow monitoring of the amount of substance expressed from the containment module.

In some embodiments of the invention, the containment module is made from material that is selected to be at least partially radiolucent or radiopaque, depending on the application.

In some embodiments of the invention, the containment module is made from material that is selected to be microwaveable, and may optionally contain a microwave susceptor to enhance heating of the containment module and/or the substance.

In some embodiments of the invention, the containment module and the substance are the same material.

In some embodiments of the invention, an individual substance delivery container is provided in a sealed package, such as a peel pack.

In some embodiments, the interior of the containment module is divided into two or more separate compartments by internal dividers, and a plurality of substances are provided that are kept separate until use.

The invention further provides a method of using the substance delivery device of the invention to deliver a substance, via a cannula, to a specified location in a living individual.

### BRIEF DESCRIPTION OF THE DRAWINGS

FIG. 1 shows an exterior view of an embodiment of a containment module of the invention.

FIG. 2 shows an exterior view of an embodiment of a containment module of the invention.

FIG. 3 shows an exterior view of an embodiment of a containment module of the invention.

FIGS. 4A-C show exterior views of embodiments of a containment module of the invention.

FIG. 5 shows an exterior view of an embodiment of a containment module of the invention.

FIGS. 6A and 6B show embodiments of the invention in which the lumen of the substance containment device is divided into more than one compartment.

FIG. 7 shows a combination of a substance delivery container and a pushing device.

FIG. 8 shows application of the invention in plugging an annulotomy.

### DETAILED DESCRIPTION OF THE INVENTION

The present invention provides a containment system for delivery of prepackaged substances to locations within a living individual that can be used in combination with existing cannulae used for this purpose.

### DEFINITIONS

While terms used in this application are generally intended to have their ordinary meaning, the following additional definitions are provided to further clarity.

(a) “cannula” refers to a tube which can be inserted into the body, often for the delivery or removal of fluid. The cannula may have a trocar attached which allows puncturing of the body to get into the intended space. The cannula may be flexible, or it may fixed in a shape consistent with an intended use. The term “cannula” as used in this application encompasses catheters, syringes and ported scopes such as GI scopes, bronchoscopes, laparascopes and arthroscopes through which a substance may be delivered.

(b) a “substance delivery container” is a combination of a containment module and a substance to be delivered via a cannula. In some embodiments, the containment module and substance are different materials (in which case the containment module has an interior lumen), while in other embodiments they are the same material.

(c) “flexible” refers to the property of the containment module that allows the substance delivery container to bend relative to an axis extending from the proximal to the distal end as needed to follow curvatures which may be present in a cannula. By way of example, it is desirable to have sufficient flexibility for the substance containment device to be able to pass through a curved cannula that has a radius of curvature of 100 feet. In some embodiments, the substance containment device has sufficient flexibility to pass through a curved cannula that has a radius of curvature of 50 feet.

(d) “compressible” refers to the property of the containment module that allows the substance delivery container to be slightly compressed with ordinary finger pressure. Such compressibility enhances the ease of handling of the substance delivery container, since it can be grasped firmly by the user for insertion in the cannula without fear of breakage.

(e) “resilient” refers to the property of the containment module or extensions of the containment module that allows the substance delivery container or extensions thereof to resist compression and to substantially reacquire an initial shape after it is bent or otherwise deformed in the course of passing through a cannula that is deflected from straight by an angle of 30° (interior angle=150°).

(f) “delivery to a defined location” refers to localized delivery within the body of an individual in a manner such that the substance delivered remains concentrated in the location for a period of time following delivery, for example a period of minutes, hours, days or even years. This is in contrast to, for example, intravenous injection, where the injected material is immediately transported through the blood system. However, delivery to a defined location does not require that the substance remain in that location permanently. The defined location may be anywhere within an individual, including without limitation boney tissues, intra-articular, intravascular (where the substance will not simply be carried away), intraspinal, intra-abdominal; in muscle, skin, or fat; within organs; on or near nerves; and within teeth

Exterior Structure of the Containment Module

In the present invention, the containment module provides the interface between the substance delivery container as a whole (containment module+substance to be delivered) and the user and the cannula. Exterior structure on the containment module therefore provides for enhanced ease of use and other enhanced features of the invention.

The containment module will commonly have a generally cylindrical shape with a defined proximal and distal ends, and a side wall. Cylindrical in this case does not indicate and particular aspect ratio, and the side wall may be larger, smaller or the same size as the ends. Furthermore, the term cylindrical does no exclude a taper such that one of the ends is bigger than the other, or such that a middle portion is larger or smaller than the ends. Moreover, the exterior of the containment module may have structure or texturing as discussed below/

FIG. 1 shows a view of an exterior view of an embodiment of a containment module of the invention. The containment module has a distal end 10 and a proximal end 12. In the proximal end 12 is a shaped receiver 14 which is circular in shape and sized to receive a tamp. Other designs for shaped receivers may be employed to conform to other devices used to drive materials through cannulae, including round, square, star, slotted and hex shapes. The dashed line in FIG. 1 shows an optional interior lumen where substance could be disposed. Shaped received may also be in the form of extensions protruding from the proximal end of the containment module that fit in a depression at the proximal end of the pushing device.

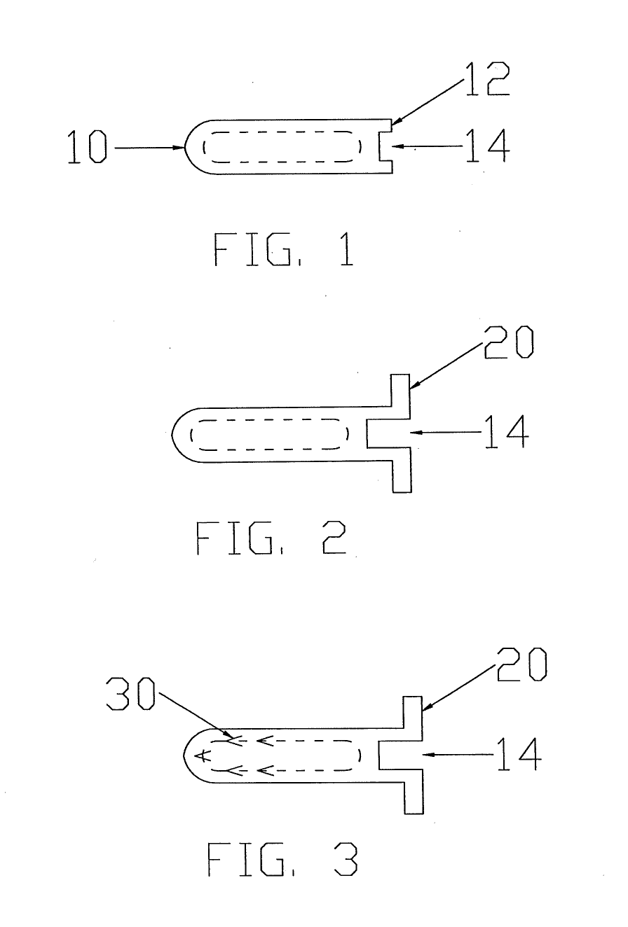

FIG. 2 shows a view of an exterior view of another embodiment of a containment module of the invention. In FIG. 2, the proximal end of the containment module has a lip 20 which is larger in diameter than containment module, and which thus extends radially outwards from the containment module, surrounding the shaped receiver 14. The lip 20 can interact with the proximal end of the cannula to prevent movement of the proximal end into the cannula.

The lip 20 may be separable from the remainder of the containment module, for example by the introduction of a score line at its connection to the remainder to the remainder of the containment module. This allows the lip to stabilize the initial position of the substance delivery container and then to be separated so that the entire substance delivery container other than the lip can be delivered via the cannula. As used herein, the term “separable” means that the containment module and lip are structured to define a particular location at which the lip separates from the containment module without damage to the containment module that would render it unsuitable for use in delivering substance via a cannula.

In other embodiments, the lip is not separable and retains the containment module at the proximal end of the cannula. In the latter case, the distal end of the containment module optionally contains predefined weak spots 30 through which the substance may be expressed as shown in FIG. 3 by pressure from the tamp or similar device. In some embodiments, the weak spot may result from changes (i.e. softening) of the material of the containment module on exposure to the conditions (temperature, wetness, pH etc) at the defined site.

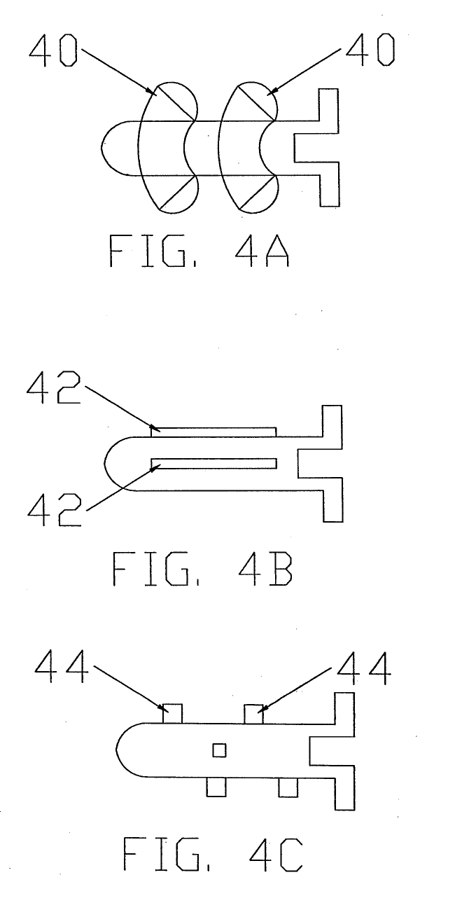

Cannulae are known and used that have differing internal diameters, for example in the range of 0.5 to 20 mm. To allow a single size of containment module to work well with a range of sizes of cannulae, the containment module may have extensions on the exterior surface thereof between the proximal end and the distal end to limit lateral movement of the containment module when inserted into cannulae having different internal diameters. FIGS. 4A-C show various embodiments of such extensions.

In FIG. 4A, one or more, for example two, resilient rings 40 extend outwards from the surface of the containment module. These rings 40 resist compression, and therefore press outwards against the interior of a cannula to stabilize the substance delivery container against lateral movement within a cannula that is larger in diameter than the body of the containment module.

In FIG. 4B, one or more ridges 42 extend outwards from the surface of the containment module and extend axially along the containment module. These ridges are compressible and can be deflected laterally to allow a fit into a variety of sizes of cannulae. In FIG. 4C flexible and compressible protrusions 44 serve this same purpose.

The rings, ridges or protrusions described above are examples of “stabilizing extensions” which may be present in embodiments of the invention. These stabilizing elements may be provided over some or all of the exterior of the containment module. Thus, at one extreme, the stabilizing extensions form a compressible layer of the entire exterior of the containment module.

Stabilizing extensions may be formed as an integral part of the containment module, i.e. from the same material, or from a different material. The stabilizing extension may be provided already attached to or as a part of the containment module, or as an attachment to be fitted on the containment module if needed for stabilization in the cannula with which the containment module is to be used. Stabilizing extensions may be removable for on-site modification to fit a particular cannula. Stabilizing extensions may be included in multiple sizes on a single containment module, with the larger ones being removed on site if needed to fit a cannula.

Some practitioners use rotation of the cannula to provide a clean separation between delivered material and material remaining in the cannula. For this to work, however, the substance delivery container cannot freely rotate within the cannula. Thus, in some embodiments of the invention the containment module has external surface structure that limits rotation of the containment module within a cannula. This function may be served by the stabilizing extensions discussed above. However, there are also structures that can be used to prevent rotation, that do not also prevent lateral movement.

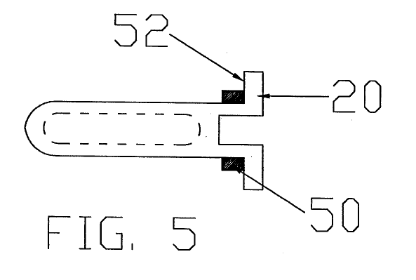

FIG. 5 shows an embodiment of the invention that incorporates a collar 50 that is roughened or tacky to limit rotation. The thickness of collar 50 is exaggerated in the figure for easy viewing. Such a collar may be formed by chemically treating or mechanically abrading the exterior of the containment module. Similar treatment might be applied to the underside 52 of the lip 20 if there is sufficient bearing surface on the proximal end of the cannula to restrict rotation.

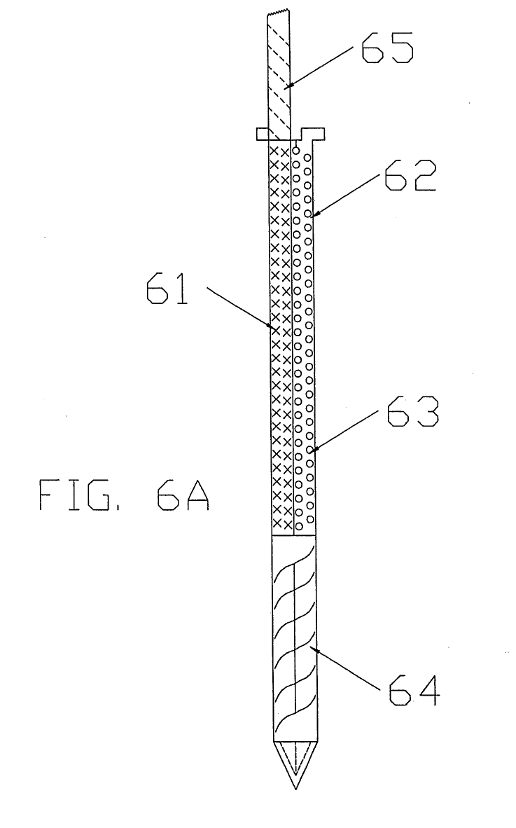

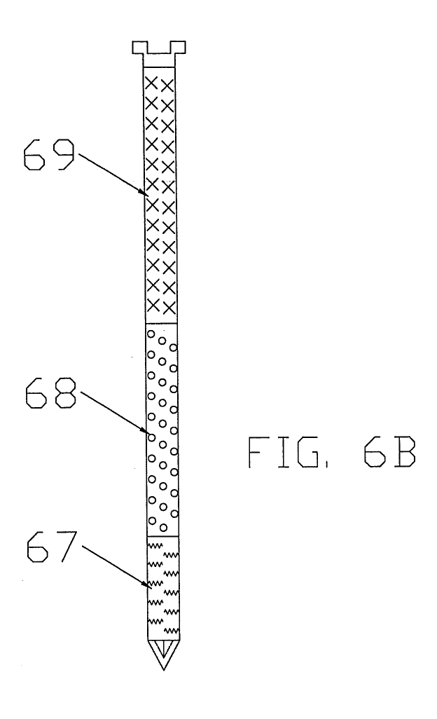

FIGS. 6A and 6B show embodiments of the invention in which the lumen of the substance containment device is divided into more than one compartment. In FIG. 6A, the lumen is divided into two lengthwise compartments 61,62 containing different substances, with a divider 63 extending the between the distal and proximal ends. Optionally a mixer, such as an Archimedes screw 64 can be provided to mix the two substances as they are driven through it be depression of plunger 65. In FIG. 6B the lumen is divided into three compartments 67, 68, 69 which would be expressed from the substance container device to a location one after the other.

Materials and Making of Containment Module

The containment module of the present invention can be made of materials of several functional types. First, the containment module can be made from a flexible material that is not intended to be introduced into the body of the living individual, but rather which remains in the cannula and has the contained substance expressed from it. Second, the containment module may be a biocompatible material that is intended for introduction into the body. Within this category are materials that (1) are left in the body and subsequently resorbed over a moderately short period of time (for example hours or days); (2) are left in the body and retain their structure for an extended period of time (for example years) to provide support and a matrix for growth; or (3) are present in the body for a period of time and subsequently removed. Different materials are suitable for making these different types of containment modules.

For example, suitable materials for use in making containment modules that will remain in the cannula are similar to those used in condoms or surgical gloves. Such materials include natural rubber latex; polyurethanes, including hydrophilic polyurethanes, collagen films such as those formed from collagen gels as described in U.S. Pat. No. 4,626,286 which is incorporated herein by reference; copolymers and blends of acrylonitrile, butadiene and styrene such as those disclosed in U.S. Pat. No. 5,284,157 which is incorporated herein by reference.

Suitable materials for use in making containment modules that will be introduced into and potentially remain in or be resorbed by the body include corn starch, autograft tissue, allograft tissue, xenograft tissue, polytetrafluorethylene (PTFE), gelatin, polyvinylchloride, polyethylene latex, natural rubbers, collagen, polylactides (see U.S. Pat. No. 6,117,928 which is incorporated herein by reference), polyglycolic acid, poly(iminocarbonates), copolymers of polyhydroxybutrate/polyhydroxyvalerate; polyalkylene oxalates, polycaprolactone, polypropylene fumarate, poly(ethyl ether) polydioxanone, and polyester-amides. Other materials are described in U.S. Pat. Nos. 5,108,755, and 6,822,000 which are incorporated herein by reference.

In some embodiments of the invention, the containment module is made to be radiopaque in whole or in part to allow radiographic visualization of the containment module during use. This may be accomplished by dispersing a radiopaque material in the containment module. In some cases, the radiopaque material is a macroscopic metallic object, such as a wire or a bead that is embedded within or located the containment module. In other instances, the radiopaque material is a more finally dispersed material, such as barium sulfate, kaolin, bentonite, talc, aluminum silicate, magnesium silicate, siliceous sand, alumina, illite, vermiculite, nontronite, saponite, chlorite, allophane, calcium phosphate, iodine, iron powder and lead powder, which can be more uniformly distributed throughout the material of the containment module.

In some embodiments of the invention, the containment module is at least partially radiolucent, while the substance is selected to be radiopaque. In this instance, the substance can be visualized as it is expressed from the containment module. Real time monitoring of the volume of substance can be maintained if the cannula used is also made of radiolucent material. In some embodiments of this application, the containment module contains radiopaque volumetric markings to further facilitate judging the quantity of substance expressed.

The containment modules of the invention are suitably made using extrusion techniques. is a common form or making containers like these. Existing medical and food packing equipment can be used to fill extruded containers with the substance to be delivered. Radio frequency can be used to seal as well as join materials uses for packing bio substance. Heat shrink is also a common packing technology that can be used. Injection molding and dip molding may also be employed depending on the specific material to be used.

For some applications, and for some practitioners, it is desirable to provide substance to the location in the living body in heated state. To facilitate this, it is desirable to have the containment module formed from a material that is microwave safe so that the substance delivery container can be heated in a microwave oven just prior to use. Subject to biocompatability requirements, it may also be desirable to incorporate microwave susceptors into the containment module to enhance heating efficiency and uniformity. “Susceptor,” for purposes of this disclosure, means any compound or material capable of converting microwaves, radio frequency transmission, or magnetic induction into heat. Such compounds include metal particles, metal oxides such as tin oxide, alkali or alkaline earth metal sulfate salts (e.g. calcium sulfate), aluminum trihydrate, quaternary ammonium salts, phosphonate compounds, phosphate compounds, and polystyrene sulfonate sodium salts as described in U.S. Pat. No. 6,649,888 which is incorporated herein by reference. Susceptors that are hydrates of inorganic salts that provide a self limiting temperature maximum as described in U.S. Pat. No. 4,283,427 which is incorporated herein by reference are also suitable.

Heating could also be performed using a water bath or a sterilizer, or an exothermic chemical reaction of a material within the substance delivery container.

In some embodiments of the invention is contemplated, the substance delivery container. may have incorporated therein an indicator to denote that a desired temperature has been reached. This could be a thermochromic material incorporated in the substance delivery container or some type of gauge associated with the substance delivery container. Where cooling is desired, this could be similarly indicated.

Substances for Use in the Substance Delivery Container

The substance delivery container of the invention can be used to contain and deliver a wide variety of different types of substances. The substance may be in a variety of forms, including without limitation liquid, powder, crystals, granules, paste or gel

In some embodiments, the substance disposed in the lumen of the containment module is a bone cement, such as an acrylic bone cement, for example a bone cement based on polymethyl methacrylate (PMMA) as is known in the art. For use with cements that require mixing of multiple components, the lumen of the containment module may be divided into multiple parts in a manner such that the components are combined upon use. (See for example, U.S. Pat. No. 7,523,103) Alternatively, the parts may be provided in separate containment modules and combined through a mixer to which substance delivery containers are attached.

The substance of the invention may also comprise bone morphogenic proteins (BMP), demineralized bone matrix (DBM), BOTOX, any bone marrow aspirate, platelet rich plasma, composite ceramic hydroxyapatite, tricalcium phosphate, glass resin mixtures, resorbable highly purified polylacttides/polylactides-co-glycolides and the like. The substance may also include therapeutics such as hormones, antibiotics, anti-cancer agents, growth factor substances, bone chips, growth matrix, whole blood or blood components, analgesics (local anesthetics, opiods, etc.), antivirals, antiparasitics, anti- or pro-thrombotic agents, thrombolytics, chemotherapeutic agents, materials for localized radiation therapy, neurolytics, chemotactic agents, cytokines, cytokine mediators, stem cells or other cellular materials (chondrocytes, osteoblasts), cell growth mediators, accelerators, or inhibitors.

Combinations of the Invention

The substance delivery container of the invention is intended to be used with a cannula, and a pushing device that interacts with the shaped receiver for driving the containment module and/or the substance contained therein through the cannula to the desired location within the living individual. Thus the invention also provides combinations of the substance delivery container with a pushing device, with a cannula, or with both. In this context, the term “combination” is used herein to refer to packaged combinations of the stated devices, or to operative combinations formed at the point of use by insertion of the substance delivery container into a cannula and/or interaction of the substance delivery container with a pushing device.

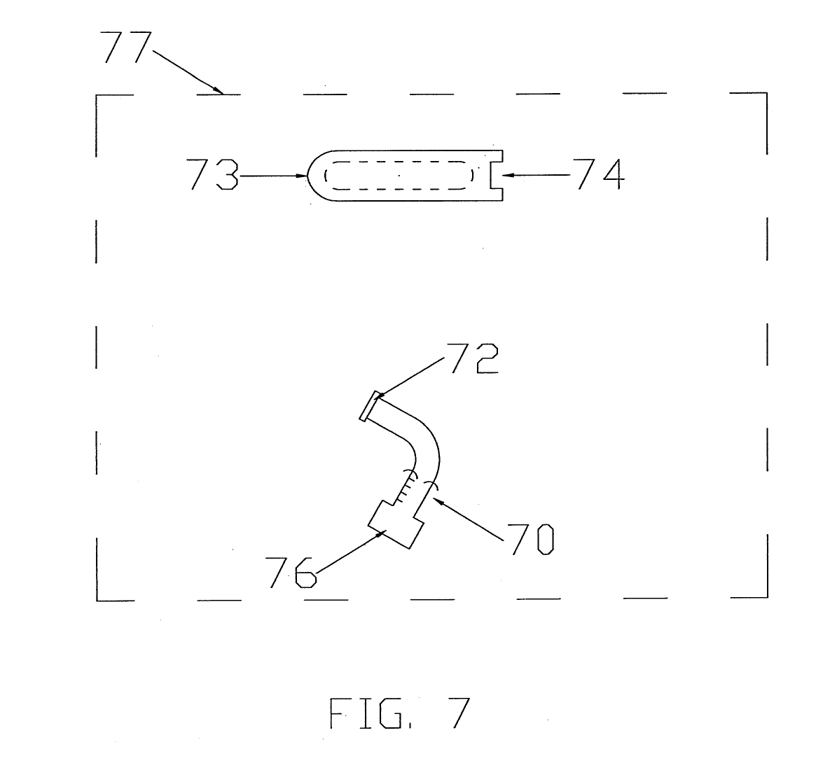

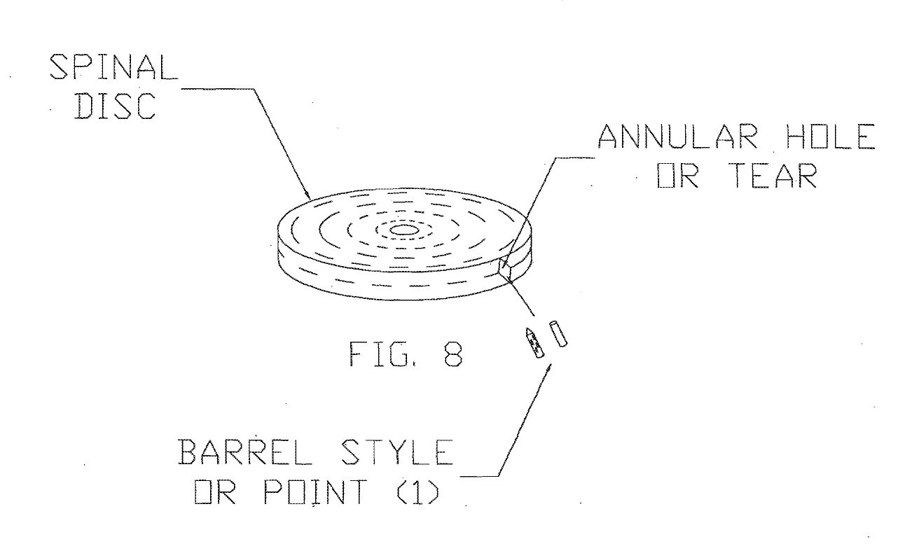

In one embodiment, the invention is a combination of a substance delivery container as described above and a pushing device, in which the pushing device has a distal end that is sized and shaped to interact with the proximal end of the substance delivery container, and an extension for control the movement of the distal end within a cannula. FIG. 7 shows such a combination, in which the pushing device 70 has a distal end 72 that fits within the shaped receiver 74 of the containment module 73. Extension 76 allows control of the pushing device to push the distal end 72 through the cannula, pushing the containment module or the substance from the containment module before it, and allowing it to be drawn back. FIG. 7 also shows optional measuring marks along the extension 76 which provide a way to monitor the amount of substance expressed or the distance that the containment module has been advanced into the cannula. The combination of the pushing device and the substance containment device are suitably packaged together in a package embodied by dashed line 77.

The proximal end of the containment module may be shaped to engage the distal end of the pushing device in a locking arrangement, for example when the pushing device is rotated relative to the containment module to be trapped under a lip of a depression, or where the distal end of the pushing device is shaped in a way that prevents free rotation (i.e. a hex or star). Such an engagement facilitates twisting or breaking off a stream of substance being expressed from the containment module and allows coordination or separation of movement between the substance deliver container and the cannula.

The combination may also include a spacer or shim for association with the plunger to conform its outside diameter more closely to the inside diameter of a larger cannula. In one embodiment, this spacer would be an annular sleeve through which the plunger can pass.

In another embodiment, the invention is a combination of a substance delivery container as described above and a cannula. The substance delivery container of the combination is sized in diameter to fit within the lumen of the cannula, although the lumen of the cannula may be larger than required to accommodate the substance delivery container, particularly where extensions to limit lateral movement are included. In the case where the substance delivery container has a lip, the size of this lip in at least one exterior dimension (if the lip is asymmetrical) is greater than the interior diameter of the cannula, and preferably equal to or greater than the exterior diameter of the cannula.

Delivery cannulae are made commerically by Kyphon, Stryker, Parralax, Nuvasice, Spine Tech, Medtronic and Zimmer. Delivery cannulae are also described in U.S. patents including U.S. Pat. Nos. 6,832,988, 6,780,191, 6,582,439, 6,887,246, 6,770,079, and 6,395,007 which are incorporated herein by reference.

In a third embodiment, the invention is a combination of a substance delivery container as described above, a pushing device and a cannula.

### METHOD OF THE INVENTION

The invention further provides a method for delivering substances to a location in a living organism. In general, the method comprises the steps of

(a) inserting a substance delivery container as described above into the proximal end of a cannula;

(b) positioning the distal end of the cannula at the location in the living organism,

(c) pushing the substance and/or the containment module through and out of the distal end of the cannula to deliver the substance and/or the containment module to the location.

Steps (a) and (b) may be performed in either order.

The characteristics of the substance delivery container and the cannula are determined by the location and the medical reasons for the delivery. In non-limiting examples, the method of the invention may be employed for introduction of bone cement, for example in connection with a vertebropasty or kyphoplasty procedure, delivery of bone cement or bone chips to fracture sites in long bones (e.g. tibial plateau or distal radius); delivery of analgesic medicines (either as liquid or within beads or on impregnated or saturated resorbable or non-resorbable solid material) to surgical sites; delivery of neurolytic agents on or near nerves for treatment of chronic pain, delivery of antibiotic beads to infected bone or abscesses, and placement of chemotherapeutic agents directly on tumors or tumor beds.

FIG. 8 shows a particular embodiment of the method invention in which the substance containment device is used to fill a surgically-created hole in boney/cartilaginous tissue. Such procedures are commonly performed in spinal disks, in a procedure referred to as an annulotomy. The substance containment device, particularly as a resorbable material, may also be used to fill a tear or injury-related hole in tissue.

1. A substance delivery container for delivery of a substance to a defined location in a living individual via a cannula, comprising:
(a) a containment module; and
(b) a substance to be delivered into a specific location of a living individual disposed within the containment module.
wherein
the containment module maintains a seal around the substance to prevent leakage and to isolate the substance from the surrounding environment prior to passage through the cannula,
the containment module is formed from a material that is flexible and compressible, and
the containment module has structure and shape sufficient to define a distal end and a proximal end, said proximal end including a shaped receiver for interacting with a device for driving the containment module and/or the substance contained therein through the cannula to the desired location within the living individual.

2. The substance delivery container of claim 1, further comprising a lip formed at the distal end of the containment module and extending radially outward therefrom.

3. The substance delivery container of claim 2, wherein the lip is separable from the containment module.

4. The substance delivery container of claim 2, wherein the lip is not separable from the containment module.

5. The substance delivery container of claim 1, further comprising an extension member extending outwards from the exterior surface of the containment module.

6. The substance delivery container of claim 1, further comprising an external surface structure that restricts rotation of the containment module within a cannula.

7. The substance delivery container of claim 1, further comprising a microwave susceptor dispersed within the containment module and/or the substance.

8. The substance delivery container of claim 1, wherein the containment module is radiopaque.

9. The substance delivery container of claim 1, wherein the containment module is radiolucent.

10. The substance delivery container of claim 1, wherein the containment module is at least partially radiolucent, and the substance is radiopaque.

11. The substance delivery container of claim 1, wherein the containment module is formed from a resorbable material.

12. The substance delivery container of claim 1, wherein the containment module and the substance are the same material.

13. The substance delivery container of claim 1, wherein the substance is a bone cement.

14. The substance delivery container of claim 1, wherein the substance is selected from the group consisting of bone chips, bone morphogenic proteins, demineralized bone matrix, BOTOX, bone marrow aspirate, platelet rich plasma, composite ceramic hydroxyapatite, tricalcium phosphate, glass resin mixtures, and resorbable highly purified polylacttides/polylactides-co-glycolides.

15. The substance delivery container of claim 1, wherein the substance is selected from the group consisting of hormones, antibiotics, anti-cancer agents, growth factor substances, analgesics, antivirals, antiparasitics, anti- or pro-thrombotic agents, thrombolytics, chemotherapeutic agents, materials for localized radiation therapy, neurolytics, chemotactic agents, cytokines, cytokine mediators, stem cells, chondrocytes, osteoblasts, and cell growth modulators.

16. A combination comprising a substance delivery container is accordance with claim 1, and a pushing device, said pushing device comprising a distal end portion that is sized and shaped to interact with the proximal end of the substance delivery container, and an extension portion attached to the distal end portion, said extension portion being of sized and shaped to control of the pushing device, thereby moving the distal end of the pushing device into and out of a cannula.

17. A combination comprising a substance delivery device in accordance with claim 1 and a cannula, wherein the substance delivery container is sized in diameter to fit within the lumen of the cannula.

18. The combination of claim 17, wherein the substance delivery container further comprises a lip formed at the distal end of the containment module and extending radially outward therefrom, and wherein an exterior dimension of said lip is greater than the interior diameter of the cannula.

19. The combination of claim 18, wherein the exterior dimension of the lip is equal to or greater than the exterior diameter of the cannula.

20. The combination of claim 17, further comprising a pushing device said pushing device comprising a distal end portion that is sized and shaped to interact with the proximal end of the substance delivery container, and an extension portion attached to the distal end portion, said extension portion being of sized and shaped to control of the pushing device, thereby moving the distal end of the pushing device into and out of the cannula.

21. A method for delivering substances to a location in a living organism comprising the steps of:
(a) inserting a substance delivery container in accordance with claim 1 into the proximal end of a cannula;
(b) positioning the distal end of the cannula at the location in the living organism, and
(c) pushing the substance and/or the containment module through and out of the distal end of the cannula to deliver the substance and/or the containment module to the location.

22. The method of claim 40, further comprising the step of heating the substance delivery container prior to insertion into the cannula.

23. The method of claim 22, wherein the substance delivery container further comprises a microwave susceptor dispersed within the containment module and/or the substance, and wherein the heating is performed by microwave irradiation.
